(19) United States
(12) Plant Patent Application Publication
(10) Pub. No.: US 2019/0239411 P1
(43) Pub. Date: Aug. 1, 2019

(54) Hosta plant named 'Blue Elf'

(50) ;

(71) Applicant: Hans A. Hansen, Zeeland, MI (US)

(72) Inventor: Hans A. Hansen, Zeeland, MI (US)

(73) Assignee: Walters Gardens Inc, Zeeland, MI (US)

(21) Appl. No.: 15/932,096

(22) Filed: Feb. 1, 2018

(51) Int. Cl. A01H 6/12

(52) U.S. Cl. PLT/353

(57) ABSTRACT

A new and distinct Hosta plant named ‘Blue Elf’ with rounded, glaucous, blue-green leaves having a near white underside. The blue-green leaf coloration remains until near the end of the growing season. The flowers are held on tightly congested scapes just above the foliage. ‘Blue Elf’ has excellent dwarf characteristics and is useful in the miniature garden, as edging or front of the landscape border, in containers, as a specimen or en masse.

Botanical classification: Hosta hybrid (Tratt.).

Variety denomination: ‘Blue Elf’.

### BACKGROUND AND ORIGIN OF THE PLANT

The present invention relates to a new and distinct hosta plant, Hosta ‘Blue Elf’ hereinafter also referred to as the new plant or by the cultivar name, ‘Blue Elf’. Hosta ‘Blue Elf’ was a cross by the inventor between ‘Blue Mouse Ears’ (not patented) as the female parent and ‘Osiris Obscur’ (not patented) as the male parent at a wholesale perennial nursery in Zeeland, Mich., USA during the summer of 2011. The new plant was assigned the breeder code 11-66-01 and passed the initial evaluation in the summer of 2015 and has been asexually propagated by division at the same nursery since 2015 and also by careful shoot tip plant tissue culture with the resultant asexually propagated plants having retained all the same traits as the original plant. Hosta ‘Blue Elf’ is stable and reproduces true to type in successive generations of asexual reproduction.

Hosta ‘Blue Elf’ has not been made publically available until it was listed for sale Jul. 10, 2017. Any public disclosure of ‘Blue Elf’ has been by the inventor, or one who obtained the material either directly or indirectly from the inventor, and any such disclosure has not been made more than one year prior to the application of this invention.

There are nearly 6,000 registered hostas with The American Hosta Society, which is the International Cultivar Registration Authority for the genus Hosta, and a similar or larger number of unregistered cultivars. Several of these have small leaf blades with slightly auriculate leaf bases and stiff, thick leaf substance. The most similar hosta cultivar known to the applicant is the female parent Hosta ‘Blue Mouse Ears’ (not patented). The male parent ‘Osiris Obscur’ has much larger habit and foliage than the new plant, and also has more rugose leaf surface with less glaucous covering. ‘Blue Mouse Ears’ is smaller in habit and in leaf blade and has less glaucous covering over the leaf blade and does not possess the slight reddish purple stippling of the petiole that the new plant has. ‘Flamenco Mouse’ (not patented) is smaller in habit, with less glaucous leaf blades, and the leaf blades also have a sinuate wave rather than the flatter leaves of the new plant. ‘Small World’ (not patented) is another small-leafed hosta that has deep green leaf blades and more narrow acute apex compared with the blue-green leaf blade broadly acute leaf apices of the new plant. ‘Bill Dress's Blue’ (not patented) has similar foliage color and shape, but the substance on the new plant is thicker, the flower scape is shorter and flowers of a brilliant violet hue with white stripes. Hosta ‘Blue Ice’ (not patented) is slower growing, has more glaucous foliage with slightly taller flower scape and light lavender flowers. Hosta ‘Blue Moon’ (not patented) is larger in habit and leaf, and the flower scape is taller, with more flowers that have a light lavender color.

Other Hosta cultivars may have small leaf blades with slightly auriculate leaf bases and stiff, thick leaf substance or other individual traits similar to ‘Blue Elf’ but the new plant differs from the above listed cultivars and all other hostas known to the applicant, by the combination of the following traits.

1. Leaves are rounded, with a glaucous blue-green surface.

2. Foliage color emerges light blue-green and holds onto the blue-green color well nearly the entire season.

3. Underside of leaf near white with heavy glaucous covering.

4. Compact habit and useful in the miniature garden, as edging or front border, in containers, as a specimen or en masse.

5. Flowers are striped with brilliant violet spots with contrasting white stripes between and densely arranged just above foliage.

### BRIEF DESCRIPTION OF THE DRAWING

The photograph of the new plant demonstrates the overall appearance of the plant, including the unique traits. The colors are as accurate as reasonably possible with color reproductions. Ambient light spectrum, temperature, source and direction may cause the appearance of minor variation in color.

The drawings shows a in a container in mid-season grown in a shaded greenhouse at a nursery in Zeeland, Mich. with supplement fertilizer and water as needed.

FIG. 1 shows the foliage and habit of the plant early in the season.

FIG. 2 shows a close-up of the outside of the flower and buds on a two-year-old plant.

FIG. 3 shows a close-up of the inside of the flower.

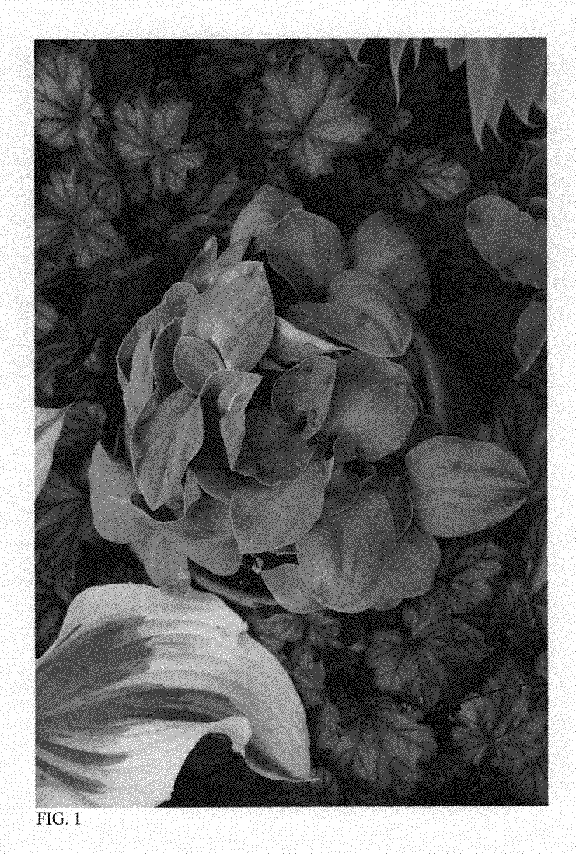

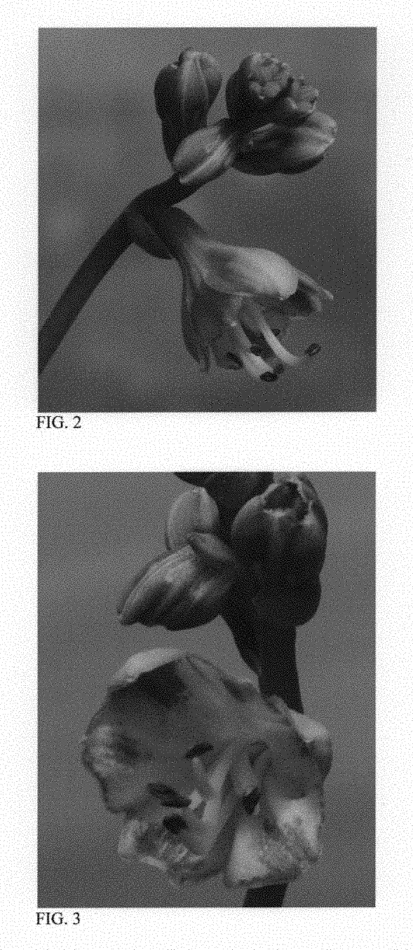

### DETAILED BOTANICAL DESCRIPTION

The following descriptions and color references are based on the 2015 edition of The Royal Horticultural Society Colour Chart except where common dictionary terms are used. The new plant, Hosta ‘Blue Elf, has not been observed under all possible environments. Those skilled in the art will appreciate that certain characteristics will vary with plants that are more mature or plants that are less mature. The phenotype may vary slightly with different environmental conditions, such as temperature, light, fertility, moisture and maturity levels, but without any change in the genotype. The following observations and size descriptions are of a potted four-year-old plant in a shaded greenhouse in Zeeland, Michigan with supplemental water and fertilizer.
Botanical classification: Hosta x hybrid;
Parentage: Female or seed parent ‘Blue Mouse Ears’—; male or pollen parent — ‘Osiris Obscur’;
Propagation: Garden division and sterile plant tissue culture;
Time to initiate roots from tissue culture: About two to three weeks;
Growth rate: Moderately vigorous;
Crop time: About three months to four months to finish during the spring in a one-liter container from rooted tissue culture plantlet;
Rooting habit: Fleshy, lightly branching;
Root color: Nearest RHS NN155C when actively growing;
Plant shape and habit: Hardy herbaceous perennial with basal rosette of leaves emerging from rhizomes producing a short, compact, mound of outwardly to slightly upwardly petioles, mostly outwardly leaves and short upwardly scapes flowering just above foliage;
Plant size: Foliage height about 10.0 cm above soil line to the top of the leaves, about 15.0 cm tall to the top of the flowers and about 28.0 cm wide at the widest point just above soil line;
Foliage description: Glabrous and glaucous both adaxial and abaxial; ovate; broadly acute apex, rounded to cordate base; margin entire; mostly flat; flexible but stiff; smooth, without blistering, dimpling or bulging;
Leaf blade size: To about 8.9 cm long and 7.6 cm wide; average about 7.8 cm long and 6.2 cm wide;
Leaf blade color: Early season and expanding adaxial blend between RHS N138A and RHS 122A, abaxial blend between RHS 188C and RHS 122C; mid-season adaxial blend between RHS 188A and RHS 122A, abaxial nearest RHS 190D; after glaucous bloom is rubbed or melted off adaxial nearest RHS 137B, abaxial RHS 138B;
Petiole: Glabrous and glaucous both adaxial and abaxial; concavo-convex proximally and more flattened distally; stiff; 6.0 cm to 9.5 cm long and 6.0 to 9.0 mm wide at base, average about 5.2 cm long and 7.5 mm wide;
Petiole color: Adaxial between RHS N138D and RHS 138B with slight red stippling at base of between RHS 183C and RHS N186C; abaxial nearest 139D;
Veins: To eleven pairs and midrib; parallel; slightly raised on abaxial side;
Veins color: Adaxial between RHS 138B and RHS 138C; abaxial veins nearest RHS N138D;
Flower description:
Buds one to two days prior to opening: Clavate with rounded apex and narrow tube base; stamens frequently exserted prior to anthesis; about 8.0 mm in diameter at widest with base narrowing at about mid-length to about 4.0 mm diameter; about 18.0 mm long;
Bud color: Nearest RHS 86D;
Flowers: Perfect; perianth shape campanulate; held nearly horizontal with pedicel at 90 degree angle to peduncle; corolla fused in basal 13.0 mm, free in the distal 11.0 mm, about 24.0 mm long and 15.0 mm wide at tepal apices, (distal flowers smaller); persists for a normal period, usually one day on plant; scapes remain effective with flowers beginning mid-July for about two weeks; with about 10 flowers per scape; distinctly second; no detectable fragrance;
Tepal: Two nearly identical sets of three, glabrous; entire margins; fused in basal 13.0 mm; clavate with broadly acute apex; each approximately 25.0 mm long and 9.0 mm wide at fusion point; corolla tube portion about 8.5 mm long and 4.0 mm diameter;
Tepal color: Abaxial coloring of both inner and outer sets nearest RHS 85A with longitudinal stripes of nearest RHS NN155C; adaxial inner set white, nearest RES NN155D with congested spotting of nearest RHS 90D in linear pattern near distal margin, adaxial outer set white nearest RHS NN155D with congested spotting of nearest RHS 90D in linear pattern near distal margin extending to longitudinal center, corolla tube portion outer color nearest RHS 85D with basal 2.0 mm nearest RHS 85C, corolla tube portion inner color nearest RHS NN155D;
Gynoecium: Single; 23.0 mm long;
   Style.—Single, terete; approximately 19.0 mm long, 1.5 mm diameter, curved upward slightly in distal 2.0 mm; color nearest RHS NN155B.
   Stigma.—Puberulent; semi-globose; about 1.0 mm in diameter; color nearest RHS NN155B.
   Ovary.—Oblong; superior; apex acute; base truncate; to about 4.0 mm long and 2.3 mm diameter in middle; color nearest RHS 145D.
Androecium: Six; to about 20.0 mm long;
   Filaments.—Six, approximately 18.0 mm long and 1.0 mm in diameter; curved upward to nearly 90 degrees in the apical 4.0 mm; color nearest RHS NN155B.
   Anthers.—Elliptic; dorsifixed, longitudinally dehiscent; ellipsoidal with rounded ends; about 3.0 mm long, 2.0 mm wide; color nearest RHS 15B.
   Pollen.—Spherical to elliptic; less than 0.1 mm long; color nearest RHS 12A.
Peduncle: Usually one per mature division; glaucous, glabrous; nearly vertical; about 15.0 cm tall, and about 4.0 mm in diameter at base; flowering portion about 3.0 cm long;
Peduncle color: Nearest RHS 138A with fine stippling to blushing nearest RHS 90D in the distal portion;
Pedicel: Short; terete; glabrous; slightly glaucous; about 2.0 mm long and 1.5 mm diameter; upright to outwardly;
Pedicel color: Nearest RHS 86D;
Floral bracts: Each flower normally subtended by a single bract; ovate to deltoid; acute apex and truncate base; entire margin; glabrous matte surface abaxial and adaxial; decreasing in size distally; average about 6.0 cm long 3.0 mm wide; drying and dehiscing before flowers open;
Bract color: Adaxial and abaxial blend nearest RHS 138A;
Fruit: Not yet observed;
Seed: Not observed; however fertility or sterility has not yet been determined;
Disease resistance: The thick leaves provide some resistance to slug feeding. Other resistance to pests and diseases common to hostas is equal that typical of other cultivars. The plant grows best and shows best coloration with plenty of moisture, adequate drainage and light shade, but is able to tolerate some drought when mature, and tolerates direct sun without leaf burn, especially during the cooler parts of the day and when provided sufficient water. Hardiness at least from USDA zone 3a through 8b, and other disease resistance is typical of that of other hostas.

It is claimed:

1. A new and distinct Hosta plant cultivar named Hosta ‘Blue Elf’ as herein described and illustrated.
